(19) United States
(12) Patent Application Publication
(10) Pub. No.: US 2005/0033228 A1
(43) Pub. Date: Feb. 10, 2005

(54) Disposable safety syringe

(76) Inventor: Hsien Tung Wang, Taipei (CN)

Correspondence Address: Hsien Tsung Wang;4 FL. No. 27, Lane 160, Hsin Sheng S. Rd., Sec.1, Taipei, 100, CN

(21) Appl. No.: 10/636,743

(22) Filed: Aug. 8, 2003

(51) Int. Cl. A61M 5/00

(52) U.S. Cl. 604/110

(57) ABSTRACT

A safety syringe comprises a barrel having an aperture in a nose, a hollow plunger in the nose, the plunger having a hole, a sealing ring, a rear inverted conic detent, and a hypodermic needle, a fastener inserted through the aperture in the hole for fastening the barrel and the plunger together, and a sliding handle having a breakable neck, an internal clip-like latch having a central groove, and a plug fastened around the latch, the plug having a front tunnel. Whereby piercing the tapered end of the detent through the tunnel into the groove for fastening the plunger and the handle together at the end of fluid dispensing, removing the fastener from the aperture and the hole for disengaging the barrel from the plunger, and retracting the handle into the barrel will allow the neck to be broken or the needle to be bent prior to bending the neck.

### BACKGROUND OF THE INVENTION

1. Field of the Invention

The present invention relates to hypodermic injection devices and more particularly to a disposable syringe with improved safety and other advantageous characteristics.

2. Description of Related Art

A conventional so-called safety syringe is shown in FIGS. 11, 12, and 13. The syringe comprises a hollow syringe barrel 60, a hollow plunger 70 disposed in a forward nose of the barrel 60, the plunger 70 including a sealing ring 71 on its surface, an inverted conic detent 72 in its rear end, and a hypodermic needle 73 in the forward end of the plunger 70, and a handle 80 slidable in the barrel 60, the handle 80 including a breakable neck 81 near its forward end, an internal clip-like latch 82 in its forward end, the latch 82 having a central groove 820, and a plastic, hollow plug 83 fastened around the latch 82, the plug 83 having a front tunnel 830 aligned with the groove 820.

Operations of the prior safety syringe are detailed below. Pull the handle 80 rearward to its maximum for drawing fluid into the barrel 60 (FIG. 11). Next, push the handle 80 forward for discharging fluid through the plunger 70 and the needle 73. The tapered end of the detent 72 pierces through the tunnel 830 into the groove 820 for fastening together at the end of fluid dispensing (FIG. 12). A medical worker then can retract the handle 80 together with the needle 73 and the plunger 70 until the needle 73 is completely retracted into the barrel 60 (FIG. 13). Finally, break the breakable neck 81 prior to throwing the syringe away. This can prevent the needle 73 from accidentally pricking a person after use.

But this is unsatisfactory for the purpose for which the invention is concerned for the following reasons: A single sealing ring 71 is insufficient to completely prevent fluid within the barrel 60 from leaking into the plunger 70. Also, it is possible that both the needle 73 and the plunger 70 retract into the barrel 60 when injecting medicine into a hard tissue of the skin through the needle 73 (i.e., high resistance condition). Alternatively, a strong push of the handle 80 may disengage the needle 73 from the barrel 60 due to pressure buildup inside the barrel 60 and the plunger 70 while dispensing fluid by a medical worker. An addition of additional one or more sealing rings 71 may solve the above problems. However, a new problem is arisen. The problem is that the locking force of the detent 72 inside the groove 820 is insufficient to overcome the sealing force of the multiple sealing rings 71. As an end, both the plunger 70 and the needle 73 cannot retract into the barrel 60 after use. Thus, the need for improvement still exists.

### SUMMARY OF THE INVENTION

It is an object of the present invention to provide a disposable, retractable safety syringe comprising a hollow syringe barrel including a forward nose and a transverse aperture in the nose; a hollow plunger in the nose, the plunger including a hole aligned with the aperture, an intermediate sealing ring on its surface, an inverted conic detent in its rear end, and a hypodermic needle in a forward end of the plunger; a fastener inserted through the aperture in the hole for fastening the barrel and the plunger together; and a handle slidable in the barrel, the handle including a breakable neck near its forward end, an internal clip-like latch in its forward end, the latch having a central groove, and a plastic, hollow plug fastened around the latch, the plug having a front tunnel aligned with the groove, whereby piercing the tapered end of the detent through the tunnel into the groove for fastening the plunger and the handle together at the end of fluid dispensing, removing the fastener from the aperture and the hole for disengaging the barrel from the plunger, and retracting the handle together with the needle and the plunger until the needle is completely retracted into the barrel will allow the breakable neck to be broken. By utilizing this disposable safety syringe, the above problems of the prior art can be overcome.

In one aspect of the present invention, the syringe further comprises a sheath having internal threads and external threads around the nose whereby completely retracting the needle into the barrel will allow the nose to be enclosed by the sheath by threadedly securing the internal threads to the external threads, the handle to be pushed to urge the needle against the bottom of the sheath for bending the needle, and the breakable neck to be broken.

The above and other objects, features and advantages of the present invention will become apparent from the following detailed description taken with the accompanying drawings.

### BRIEF DESCRIPTION OF THE DRAWINGS

FIG. 1 is a perspective view of a first preferred embodiment of disposable safety syringe according to the invention;

FIG. 2 is an exploded view of the syringe shown in FIG. 1;

FIGS. 3, 4 and 5 are cross-sectional views of the syringe for illustrating positions thereof prior to dispensing, after dispensing, and after retracting the needle into the barrel respectively;

FIG. 6 is a perspective view of a second preferred embodiment of disposable safety syringe according to the invention, where the sheath is separated for illustration purpose;

FIGS. 7 and 8 are cross-sectional views of the syringe shown in FIG. 6 for illustrating positions thereof after dispensing and after retracting the needle into the barrel respectively;

FIGS. 9 and 10 are cross-sectional views of the syringe shown in FIG. 6 for illustrating positions thereof after the sheath has been secured to a forward nose of the syringe after fluid dispensing with the needle being bent and after retracting the bent needle into the barrel and breaking the handle respectively; and

FIGS. 11, 12 and 13 are cross-sectional views of a prior safety syringe for illustrating positions thereof prior to dispensing, after dispensing, and after retracting the needle into the barrel respectively.

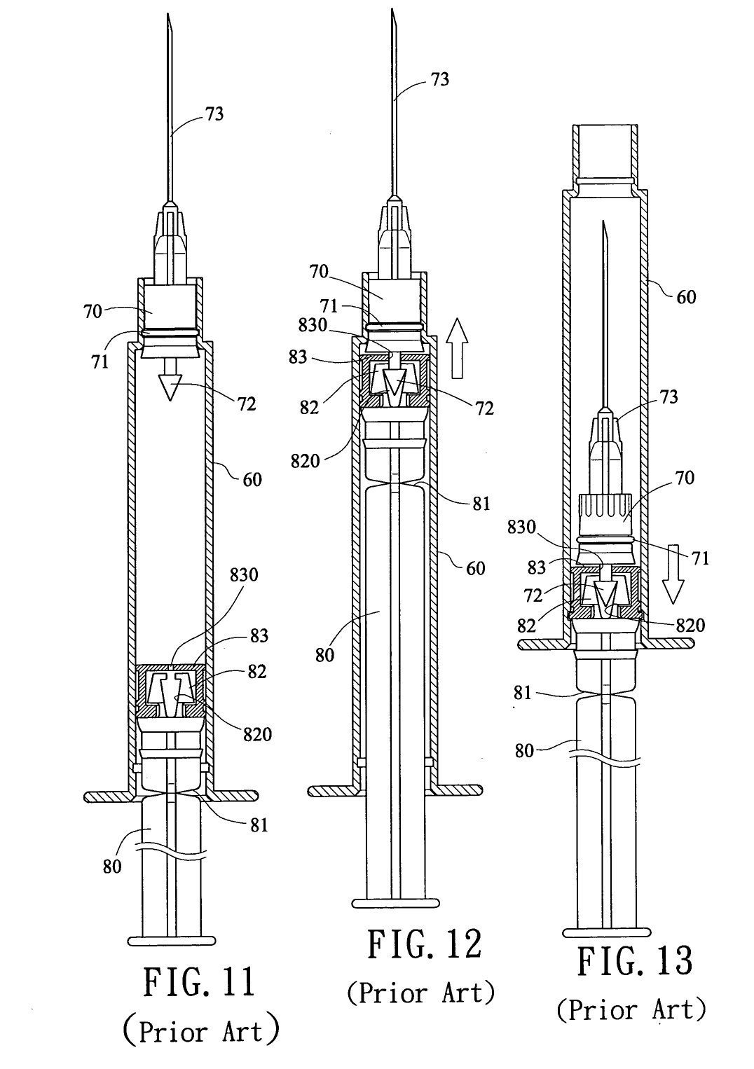

### DETAILED DESCRIPTION OF THE PREFERRED EMBODIMENTS

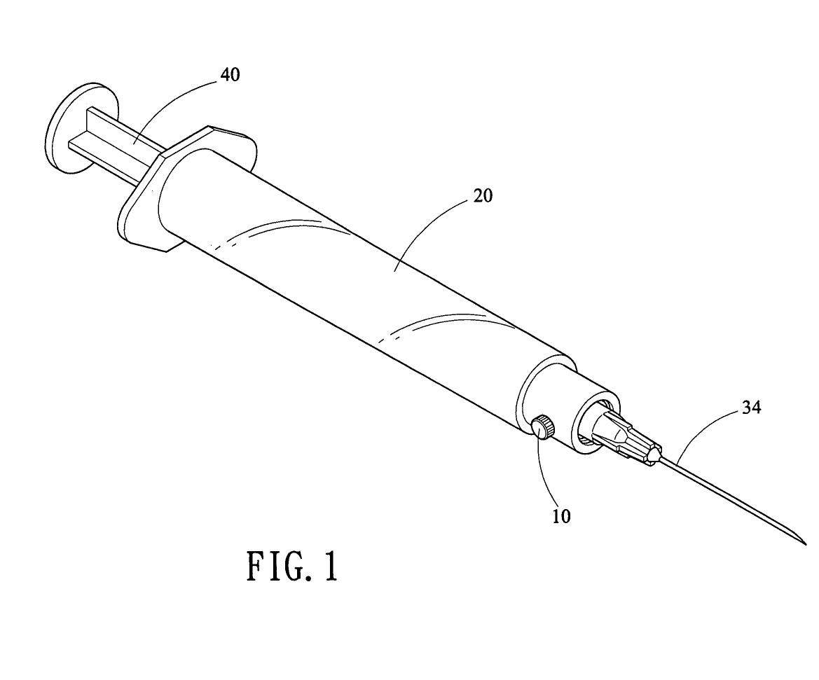

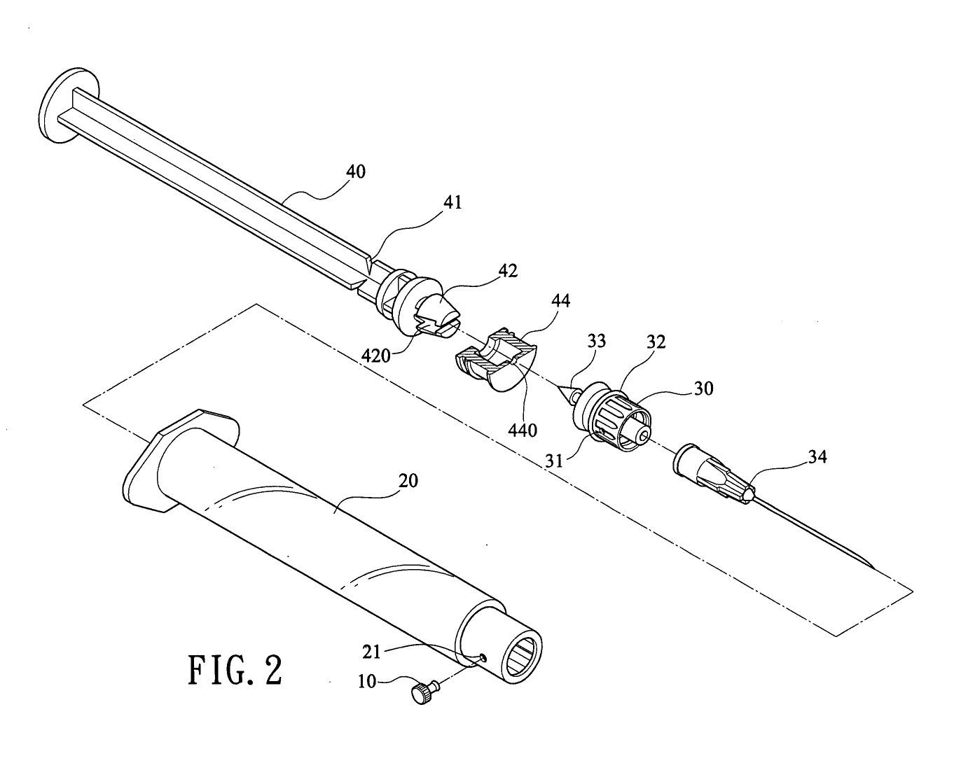

Referring to FIGS. 1 to 5, a disposable safety syringe constructed in accordance with a first preferred embodiment of the invention is shown. The syringe comprises a hollow syringe barrel 20 including a transverse aperture 21 in a forward nose, a hollow plunger 30 disposed in the nose of the barrel 20, the plunger 30 including a hole 31 aligned with the aperture 21, an intermediate sealing ring 32 on its outer surface for completely preventing fluid within the barrel 20 from leaking into the plunger 30, an inverted conic detent 33 in its rear end, and a hypodermic needle 34 in the forward end of the plunger 30, a pin 10 having an enlarged head in this embodiment, i.e., the head being larger than both the aperture 21 and the hole 31 (or a threaded shank in any of other embodiments) being adapted to insert through the aperture 21 in the hole 31 for fastening the barrel 20 and the plunger 30 together, and a handle 40 slidable in the barrel 20, the handle 40 including a breakable neck 41 near its forward end, an internal clip-like latch 42 in its forward end, the latch 42 having a central groove 420, and an elastic plastic, hollow plug 44 fastened around the latch 42, the plug 44 having a front tunnel 440 aligned with the groove 420.

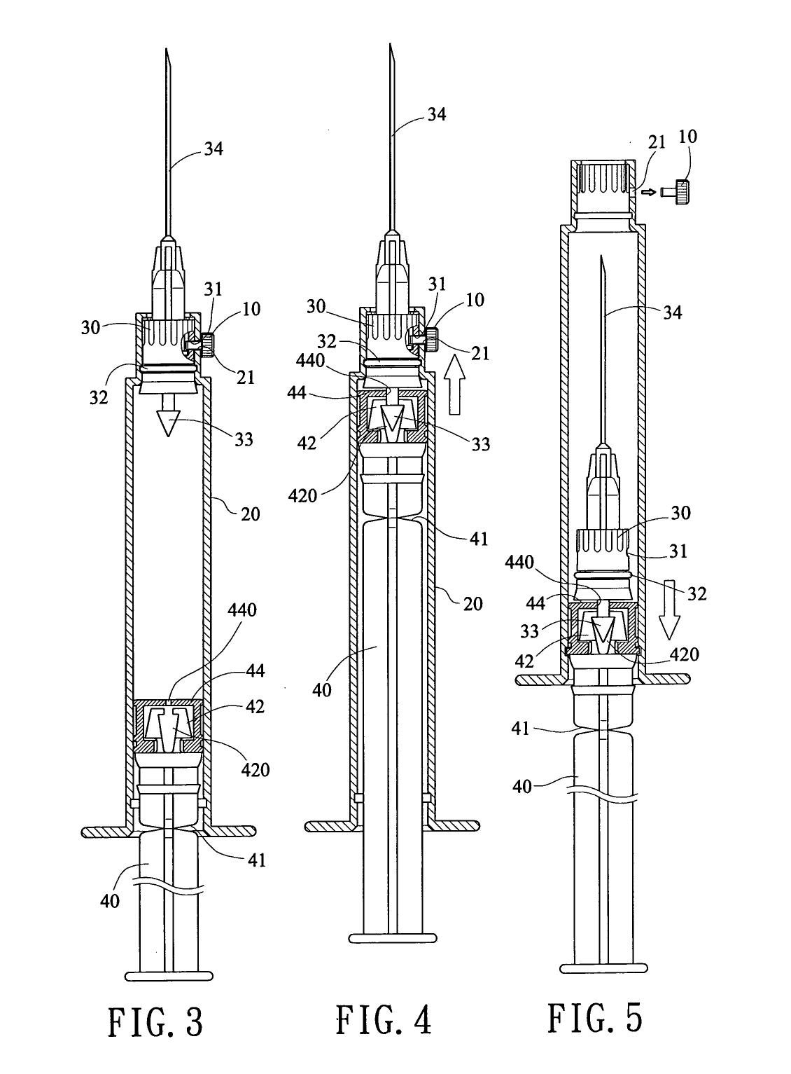

Operations of the safety syringe will be described in detail below. Pull the handle 40 rearward to its maximum for drawing fluid into the barrel 20 (FIG. 3). Next, push the handle 40 forward for discharging fluid through the plunger 30 and the needle 34. The tapered end of the detent 33 pierces through the tunnel 440 into the groove 420 for fastening together at the end of fluid dispensing (FIG. 4). A medical worker then can remove the pin 10 from the aperture 21 and the hole 31 for disengaging the barrel 20 from the plunger 30 prior to retracting the handle 40 together with the needle 34 and the plunger 30 until the needle 34 is completely retracted into the barrel 20 (FIG. 5). Finally, break the breakable neck 41 prior to throwing the syringe away. This can prevent the needle 34 from accidentally pricking a person after use.

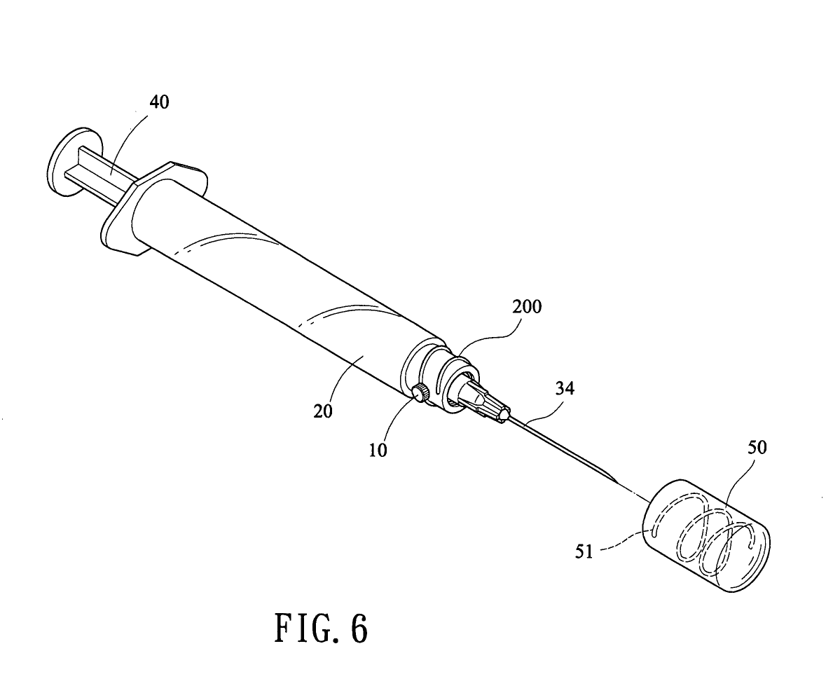

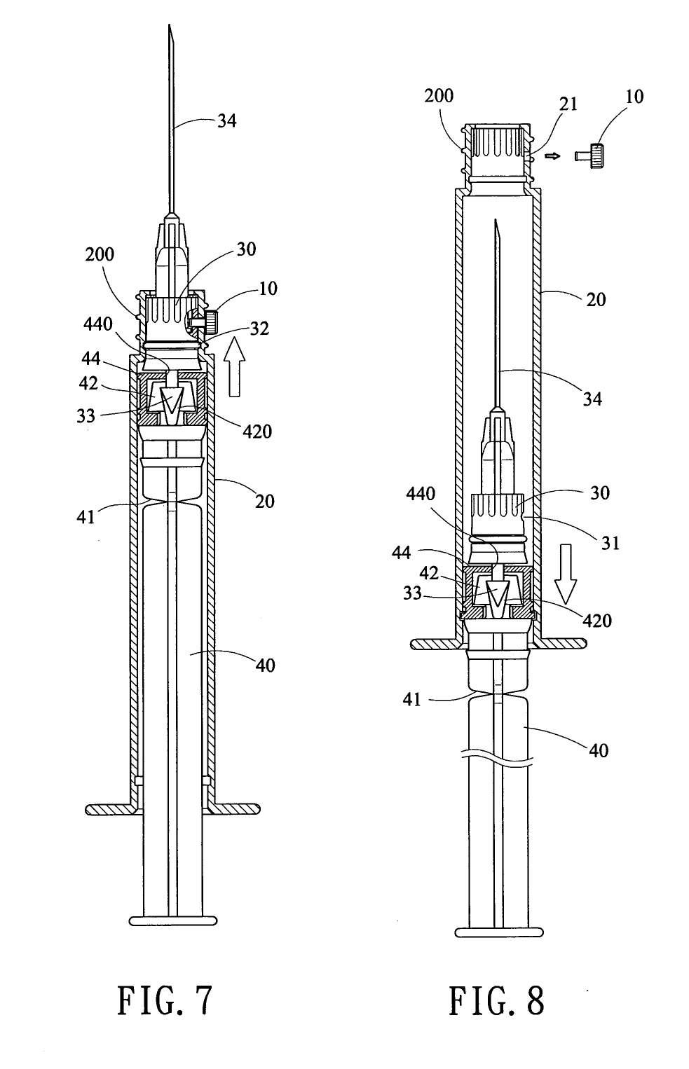

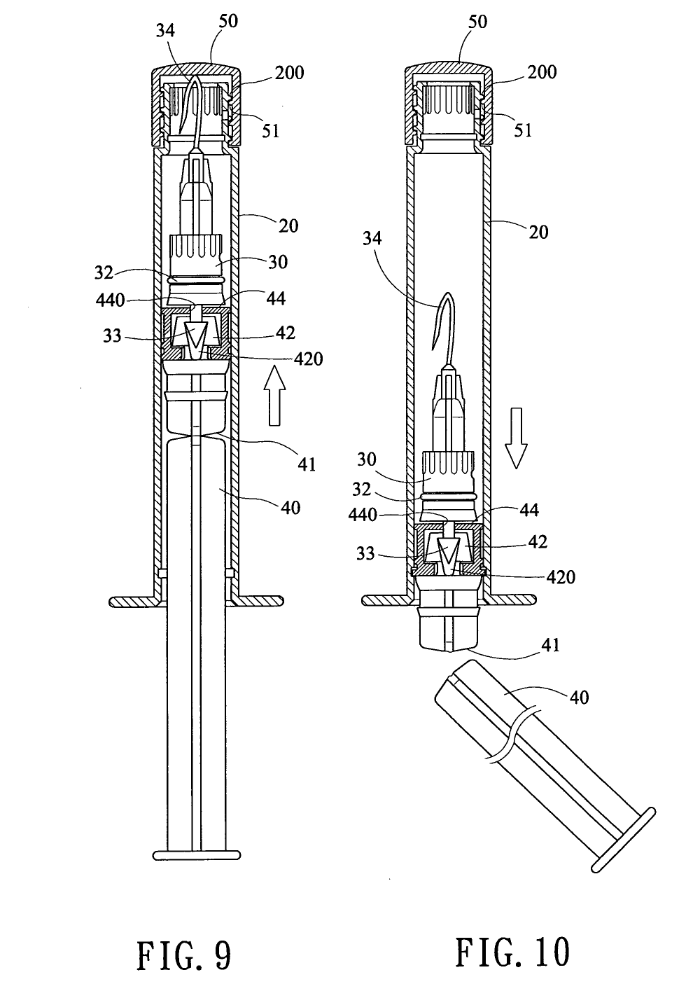

Referring to FIGS. 6 to 10, a disposable safety syringe constructed in accordance with a second preferred embodiment of the invention is shown. The second preferred embodiment substantially has same structure as the first preferred embodiment. The differences between the first and the second preferred embodiments, i.e., the characteristics of the second preferred embodiment are detailed below. A sheath 50 having internal threads 51 is provided. Correspondingly, external threads 200 are formed around the nose of the barrel 20 (see FIGS. 6 and 7). After the needle 34 has completely retracted into the barrel 20, a medical worker can enclose the nose of the barrel 20 by the sheath 50 by threadedly securing the internal threads 51 to the external threads 200. Next, the medical worker can push the handle 40 forward to urge the needle 34 against the bottom of the sheath 50 in order to bend the needle 34 to a maximum, i.e., the needle 34 is damaged (see FIG. 9). Finally, break the breakable neck 41 prior to throwing the syringe away (see FIG. 10). This can prevent the needle 34 from accidentally pricking a person after use.

While the invention herein disclosed has been described by means of specific embodiments, numerous modifications and variations could be made thereto by those skilled in the art without departing from the scope and spirit of the invention set forth in the claims.

1. A disposable, retractable syringe comprising:
a hollow syringe barrel including a forward nose and a transverse aperture in the nose;
a hollow plunger in the nose, the plunger including a transverse hole aligned with the aperture, an intermediate sealing ring on its outer surface, an inverted conic detent in its rear end, and a hypodermic needle in a forward end of the plunger;
a fastener inserted through the aperture in the hole for fastening the barrel and the plunger together; and
a handle slidable in the barrel, the handle including an internal clip-like latch in its forward end, the latch having a central groove, and an elastic plastic, hollow plug fastened around the latch, the plug having a front tunnel aligned with the groove,
whereby piercing the tapered end of the detent through the tunnel into the groove for fastening the plunger and the handle together at the end of fluid dispensing, removing the fastener from the aperture and the hole for disengaging the barrel from the plunger, and retracting the handle together with the needle and the plunger until the needle is completely retracted into the barrel.

2. The syringe of claim 1, wherein the fastener is a pin having a head larger than both the aperture and the hole.

3. The syringe of claim 1, wherein the fastener is a pin having a threaded shank.

4. The syringe of claim 1, wherein the handle further comprises a breakable neck on its forward end, whereby when the needle is completely retracted into the barrel, the breakable neck will allow to be broken.

5. The syringe of claim 1, further comprising a sheath having internal threads and the nose having external threads therearound whereby completely retracting the needle into the barrel will allow the nose to be enclosed by the sheath by threadedly securing the internal threads to the external threads on the nose, wherein the needle is to be pushed to urge by the handle against the bottom of the sheath for bending the needle.

6. The syringe of claim 5, wherein the handle further comprising a breakable neck on its forward end, whereby when the needle is completely retracted into the barrel, the breakable neck will allow to be broken.
